(19) United States
(12) Patent Application Publication
(10) Pub. No.: US 2026/0144561 A1
(43) Pub. Date: May 28, 2026

(54) SURGICAL JAW PART TOOL, SURGICAL INSTRUMENT, AND ROBOTIC SURGICAL INSTRUMENT SYSTEM

(71) Applicant: KARL STORZ SE & Co. KG, Tuttlingen (DE)

(72) Inventor: Sven Axel GRUENER, Tuttlingen (DE)

(21) Appl. No.: 19/395,717

(22) Filed: Nov. 20, 2025

(30) Foreign Application Priority Data
Nov. 22, 2024 (DE) 10 2024 134 411.3

(51) Int. Cl. A61B 17/29; A61B 34/30

(52) U.S. Cl. CPC A61B 17/29; A61B 34/30; A61B 2017/2926

(57) ABSTRACT

The present invention provides a bipolar surgical jaw part tool (8) and a bipolar surgical instrument (50) created therewith. The jaw part tool (8) has two, pivotally mounted arms (2, 2′), which have an electrically conductive material, and a force transmission means (9), which is operatively connected to the arms (2, 2′) and is designed to provide the instrument (50) by coupling to an instrument shaft (51) and to an actuating means (52), wherein the arms (2, 2′) are arranged on the distal side of the instrument shaft (51), and the force transmission means (9) is arranged in the instrument shaft (51) so as to be movable along a longitudinal axis (L) and capable of being brought into engagement with the actuating means (52) on the proximal side of the instrument shaft (51). The jaw part tool (8) has a jaw part core (1) made of an electrically insulating material and two recesses (10, 10′) separated from one another by a core wall (1′), each providing a joint axis (A, A′) which divides the recesses (10, 10′) into an exit and an actuation pivot region (13, 14). Each arm (2, 2′) has a mounting portion (23) between a jaw part portion and an actuating portion (21, 22), on which mounting portion the arm (2, 2′) is mounted in the recess (10, 10′) so as to be pivotable around the respective joint axis (A, A′), wherein the jaw part portion (21) extends through the exit pivot region (13) and out of the recess (10, 10′), and the actuating portion (22) is arranged in the actuation pivot region (14) of the recess (10, 10′). The jaw part tool (8) has two control tabs (3, 3′), which are connected to the force transmission means (9) and each extend into the actuation pivot region (14) of the recesses (10, 10′) and there engage with the actuating portion (22) of the respective arm (2, 2′). Furthermore, a robotic surgical instrument system is disclosed.

### SUMMARY

The invention relates to a bipolar surgical jaw part tool for a bipolar surgical instrument and to a bipolar surgical instrument having such a jaw part tool. The invention further relates to a robotic surgical instrument system having such a bipolar surgical instrument.

It is known from the prior art to use bipolar surgical instruments with a jaw part tool consisting of movable arms, such as dissecting and grasping forceps and scissors, which, on the one hand, have a dissecting, grasping, or cutting function depending upon the shape and size of the arms and, on the other, have a coagulation function when bipolar current is applied to the arms. To arrange the tool at a distal end of a shaft connected to an actuating means (e.g., handle, actuator, or interface to a robot system), the instrument has a tool insert with a jaw part mechanism for opening and closing the arms. The jaw part mechanism comprises at least one joint, around the joint axis of which at least one of the two arms can be moved. A distinction is made between single-sided opening jaw part tools, where only one arm is moved, and double-sided opening jaw part tools, where both arms are moved. In this application, double-sided opening jaw part tools are preferred. Using bipolar energy, current can be allowed to flow in a controlled manner between the two arms, so that tissue located between the arms is coagulated. Since the arms also function as electrodes, they have an electrically conductive material to form the electrodes.

In order to avoid short circuits between the arms, they are usually not made entirely of the electrically conductive material, but have an insulating layer at least in the region of the joint or are constructed in several parts with insulating portions. Such insulators made of electrically non-conductive plastics or ceramics usually have a dielectric strength of 10 to 50 kV/mm, so that direct breakdown through the insulator hardly occurs, since a layer thickness of just one tenth of a millimeter is sufficient to separate at least 1,000 V from one another, and mechanical strength and manufacturing usually require wall thicknesses of at least two tenths of a millimeter anyway.

However, another type of failure is critical for bipolar instruments, viz., a breakdown along the surface of the insulator along a so-called creepage stretch. This can be remedied by increasing the geometric distance between the arms in order to make the creepage stretch along the insulator surface as long as possible, so that breakdown occurs only at higher voltages. However, because conductive liquids (blood, saline, etc.) promote breakdown during use, and they accumulate in particular in covered regions of the jaw part mechanism, internal creepage stretches are even more problematic. Apart from the distance, the material of the insulator influences susceptibility to the formation of creepage stretches. The comparative tracking index of an insulating material is measured with the CTI (comparative tracking index) value. For this purpose, 50 drops of standardized electrolyte solution are dripped onto the insulator surface between two electrodes at a predetermined distance, and the voltage is measured with the electrodes on the insulator surface, up to which voltage the insulator shows no tracking or at which it becomes conductive.

High CTI values of the insulating material are advantageous for a good comparative tracking index. However, there are mechanical requirements for the material, which is why plastics with a low CTI value of 100 to 150, such as PEEK, are sometimes used as insulators. This is because plastics with a higher CTI value are often too soft for the mechanical requirements, and ceramics are too brittle.

DE 102 36 070 A1 discloses a jaw part mechanism of medical forceps. The proximal end of the movable jaw parts has at its proximal end an articulated arm, which is guided in a guide track.

Published patent application US 2006/0173452 A1 discloses a bipolar surgical instrument for closing vessels. A stop ensures a minimum distance between the two movable jaw parts.

To date, none of the known bipolar jaw part instruments for surgical applications meets all the requirements placed upon them, which include high application voltage, mechanical strength, durability, good cleanability, and, ultimately, low component and manufacturing costs.

Based upon this prior art, it is the object of the present invention to provide an improved jaw part tool for a bipolar surgical instrument.

This object is achieved by a bipolar surgical jaw part tool having the features of claim 1.

The further object of providing a correspondingly improved bipolar surgical instrument is achieved by the bipolar surgical instrument having the features of independent claim 15.

The still further object of providing a correspondingly improved robotic surgical instrument system with a bipolar surgical instrument is achieved by the robotic surgical instrument system having the features of independent claim 16.

Further developments or preferred embodiments are set out in the subclaims.

According to a first embodiment, a bipolar surgical jaw part tool according to the invention, which is designed to provide a bipolar surgical instrument by coupling to an instrument shaft and to an actuating means, has two arms pivotally mounted for opening and closing, and a force transmission means. The two arms, which can be arranged at a distal end of the instrument shaft when forming the bipolar surgical instrument, have an electrically conductive material to provide electrodes. They are, for mechanical and electrical control, operatively connected to the force transmission means. During formation of the instrument, the force transmission means can be arranged in the instrument shaft so as to be movable along a common longitudinal axis and can be engaged with the actuating means at a proximal end of the instrument shaft. According to the invention, the jaw part tool has a jaw part core consisting of an electrically insulating material and having two recesses separated from one another by a core wall. Both recesses are designed to each provide a joint axis for the arms, which divides the recesses into an exit pivot region and an actuation pivot region. Each arm further has, between a jaw part portion and an actuating portion, a mounting portion, where each arm is mounted in the respective recess so as to be pivotable around the joint axis. The jaw part portion extends through the exit pivot region and out of the recess, and the actuating portion is arranged in the actuation pivot region of the recess. The jaw part tool also has two control tabs, which act as elongated, flat connecting pieces that provide the operative connection between the arms and the force transmission means. For this purpose, the control tabs are connected at one end to the force transmission means and extend at the other end into the actuation pivot region of the recesses and engage with the actuating portion of the respective arm arranged therein.

In the jaw part tool according to the invention, the jaw part core not only ensures the mechanical mounting of the two double-sided opening arms, but also ensures safe electrical insulation of the two arms. Furthermore, the functions for mechanical and electrical control of both arms are combined in the control tabs. In the limited installation space for surgical tools, in particular also for tool diameters of 5.5 mm or less, e.g., 3.5 mm, the jaw part tool achieves a high level of mechanical stability through the joint interaction of the arms mounted in the jaw part core and connected to the control tabs.

The mechanism provided by the jaw part core has favorable force flows that allow the use of electrically non-conductive materials with high CTI values, in particular ceramics or plastics such as polybutylene terephthalate (PBT) with a CTI of 500, polyethylene (PE-LD, PE-HD), polyester resin, polytetrafluoroethylene (PTFE), polyvinylidene fluoride (PVDF), each with a CTI of 600.

A “force transmission means” is understood to mean elongated apparatuses that are suitable for transmitting a translational force or movement, generated by the proximal actuating means, through the instrument shaft to the distal jaw part mechanism, such as rigid or flexible push-pull rods. In a flexible force transmission means, the longitudinal axis refers to an extended arrangement of the force transmission means. Directional information relating to the longitudinal axis applies to flexible force transmission means not only in the straight arrangement, but also in a correspondingly modified manner, when the flexible force transmission means is in a curved arrangement. For example, movements of the force transmission means along the longitudinal axis are more generally to be understood as back-and-forth movements of the force transmission means within the instrument shaft, i. e., also in case of a curved course.

Manually operated handles, actuators, or, alternatively, robotic actuating units can be used as actuating means of a surgical instrument.

As regards the electrically conductive materials and electrically insulating materials of the various components of the jaw part tool according to the invention, the following is noted: The electrically conductive materials of the electrically connected components, such as the aforementioned arms, control tabs, and force transmission means, and all electrically connected components listed below, can be the same electrically conductive material or different electrically conductive materials, which the person skilled in the art knows how to select according to the respective requirements. The same applies in a similar manner to the electrically insulating materials of the aforementioned jaw part core and all electrically insulating components listed below. This means that the various electrically insulating components can have the same electrically insulating material, or different electrically insulating materials can be used depending upon the requirements placed upon the respective component.

According to a further embodiment, the jaw part tool according to the invention can have a fork holder which secures the mounting of the arms on the jaw part core against loosening. The fork holder is made of electrically insulating material and has on the proximal side at least one tube portion, which is provided and designed to be arranged on at least one tube portion of the jaw part core. The fork holder has two holding portions on the distal side which are designed to positively grip the jaw part core around the recesses and to secure the engagement of the control tabs with the actuating portions of the arms as well as their articulated mounting on the mounting portions in the respective recesses.

In order to positively grip the jaw part core, the holding portions define an inner contour which is formed in a region around the recesses in accordance with the outer contour of the jaw part core with the therein-arranged portions of the arms and control tabs. The positive grip is achieved by the contact between the inner surfaces, orthogonal to the joint axes, of the two holding portions and the outward-facing surfaces of the jaw part core, the arms, and the control tabs in the region around the recesses. In this way, the holding portions block a release movement of the arms and control tabs from the recesses in a direction away from the longitudinal axis and parallel to the joint axis.

According to a further embodiment of the jaw part tool according to the invention, the joint axes of the arms in the recesses of the jaw part core are each formed by a joint pin rotatably mounted in a joint bushing. Preferably, the joint bushings are formed in the recesses and the joint pins on the arms, so that the joint bushings protrude into the core wall, and the arms can advantageously be manufactured in one piece with the joint pins. Alternatively, it is also possible for the joint pins to be connected to the arms as separate axle elements, or for the joint pins to be formed at the recesses, as a projection on the core wall, and for the joint bushings to be formed in the arms. Here too, the joint pins can be integral with the jaw part core or exist as separate axle elements.

The recesses, preferably together with the joint bushings (alternatively, joint pins), are formed to be rotationally symmetrical, with respect to the longitudinal axis, on the jaw part core on opposite sides of the core wall, so that both arms can be identical. The two joint axes, which run orthogonally to the longitudinal axis, are radially spaced therefrom, so that, in the rotationally symmetrical arrangement of the recesses, they are not aligned with one another, and no connection can be established between the recesses.

Alternatively, it is also possible for one of the recesses to have a joint bushing and for the other recess to have a joint pin to form the joint axes, so that one of the arms has a joint pin, and the other one has a joint bushing. Although this embodiment requires more complex manufacturing, it can be advantageous for a tool with different arms if each arm can be arranged only in the corresponding recess.

According to a further embodiment of the jaw part tool according to the invention, it is provided for the engagement of the control tab with the actuating portion of the arm to be provided by an actuating pin received in a mounting opening, which actuating pin defines an actuating axis that is spaced from the joint axis and parallel thereto. Preferably, the actuating pin is here also formed on the actuating portion of the arm, and the mounting opening is formed in the control tab. Here, too, integral production of the actuating pin with the arm is preferred, but it is also possible to use a separate axle element as an actuating pin, which is connected to the arm. Alternatively, the mounting opening can be formed in the actuating portion of the arm, and the actuating pin can be formed on the control tab.

The engagement of the actuating pin in the mounting opening ensures the movement for opening and closing the jaw part portions of the arms and at the same time for the transmission of the electrical potential. The distance of the actuating axis from the joint axis determines the torque for rotating the arm around the joint axis and is selected as large as possible to achieve a favorable lever arm ratio to the jaw part portion. Placing the joint axis in the recess as close as possible to the edge of the jaw part core allows maximizing the distance of the actuating pin at the actuating portion from the joint axis within the available installation space.

Therefore, in a preferred embodiment of the jaw part tool according to the invention, the actuating pin at the actuating portion and the joint pin at the mounting portion of the respective arm can be arranged pointing in opposite directions.

According to yet another embodiment of the jaw part tool according to the invention, each arm can be made in one piece from the electrically conductive material without insulating layers or insulating portions. The jaw part portion has at least one functional surface for preparing, grasping, or cutting, and for electrocoagulation. And the actuating portion is made of an electrically conductive material for planar contact with a planar contact portion of the control tab. The respective contact surfaces of the actuating portion and contact portion are orthogonal to the joint axis, so that they are pressed against one another by the holding portions of the fork holder, and the electrical contact resistance is particularly low.

According to a further embodiment, it is further provided for the force transmission means of a jaw part tool according to the invention to have a sleeve for connecting to the control tabs. The force transmission means is divided into a first portion, which is a distal, uninsulated end portion, and into a second, circumferentially insulated portion, which adjoins the distal end portion and is offset in diameter on the proximal side from a third, circumferentially insulated portion of the force transmission means. The sleeve is arranged at the second portion so that the sleeve is electrically separated from the force transmission means by the circumferential insulation, and extends to the third portion, wherein the sleeve is mechanically connected to the force transmission means. A first control tab is electrically and mechanically connected to the distal, uninsulated end portion. The second control tab, which is longer than the first control tab, is electrically and mechanically connected to the sleeve.

According to yet another embodiment of the jaw part tool according to the invention, the at least one tube portion of the jaw part core can have a longitudinal bore into which the force transmission means extends in a longitudinally movable manner, so that the distal end portion of the force transmission means lies in the longitudinal bore.

In a further development thereof, the jaw part core of a jaw part tool according to the invention has a guide profile for each control tab, in which one of the control tabs is guided and moved by the force transmission means in a direction which lies in a plane with the longitudinal axis. A first guide profile is designed to guide the first control tab connected to the distal end portion of the force transmission means. For this purpose, the first guide profile extends from the actuation pivot region of one of the recesses along the at least one tube portion. The first guide profile ends in front of a proximal end of the at least one tube portion and opens into the longitudinal bore. The second guide profile, which is designed to guide the second control tab that is connected to the sleeve, extends from the actuation pivot region of the other recess to the proximal end of the at least one tube portion.

According to yet another embodiment, the jaw part tool according to the invention has a shaft connector sleeve, which is connected to the jaw part core on the proximal side and has at least one shaft connecting element for connecting to the instrument shaft.

In a preferred further development of said jaw part tool according to the invention, the shaft connector sleeve can be designed for arrangement on a proximal tube portion of the fork holder, which is arranged on a proximal tube portion of the jaw part core. The shaft connector sleeve and the proximal tube portions of the fork holder and jaw part core each have a radially aligned, mutually aligned locking opening in which a locking element is arranged that connects the shaft connector sleeve and the fork holder to the jaw part core.

Further embodiments of the jaw part tool according to the invention relate to fastening variants of the fork holder: In one embodiment, for example, the two holding portions of the fork holder can each have an opening, which corresponds in terms of shape and dimensions to a collar, which is designed for long creepage stretches on the jaw part core adjacent to the respective recess, preferably distally adjacent to the recess. In order to allow the holding portions when pushed onto the jaw part core to spread over the collars until they snap into place with the openings on the collars, the holding portions are elastically deformable in the radial direction to the longitudinal axis L. For this purpose, for example, the electrically insulating material of the fork holder can be provided at least in the region of the holding portions by an elastically deformable plastic material, and/or each holding portion can have a portion with a reduced wall thickness. Alternatively, when using a non-elastic material such as ceramic, the fork holder can be made up by two identical holder shells or can be divided lengthwise into two holder shells, each with one of the holding portions. The two holder shells that positively enclose the jaw part core can be held together on the proximal side by a sleeve-for example, the shaft connector sleeve. The gap between the two holder shells is electrically non-critical, since it runs transverse to the relevant creepage stretches in the longitudinal direction.

In order to hold the two holder shells together also on the distal side, or to secure the elastically deformable holding portions of a one-piece fork holder, the fork holder can, in further embodiments, be connected to the jaw part core at the two holding portions by at least one cylindrical connecting element, or materially bonded by a (plastic) welded or adhesive connection. A further alternative or additional embodiment relates to a material bond between the at least one tube portion of the fork holder and the jaw part core by a (plastic) welded or adhesive connection.

Fixation by connecting element(s) can advantageously be designed to be releasable so that the jaw part tool can be completely disassembled after use and reused after cleaning and disinfection. The material bonds between the fork holder and the jaw part core advantageously ensure extensive sealing of the recesses. This largely prevents creepage stretches between the control tabs.

The distal-side fixation of the fork holder to the two holding portions with one or two cylindrical connecting element(s) is preferably carried out on the collars, which encompass the holding portions with their opening. The collars then define either a through hole for a continuous cylindrical connecting element, such as a rivet, or a blind hole for two individual connecting elements, such as screws.

Since a continuous connecting element is associated with the formation of a creepage stretch, a seal can also be provided to prevent the penetration of conductive liquids. Fixation with separate connecting elements, such as screws, does not require a through hole and therefore avoids the formation of such a creepage stretch, even without sealing. Also, screws advantageously allow the holding force of the holding portions to be increased and a tightening torque to be selected in order to improve the lateral hold of the arms, which is important in particular for scissor tools whose jaw part portions must be guided tightly laterally for a clean separation of tissue.

In a further embodiment, it can also be provided for the connecting elements used to terminate at the head end with an outer surface of the holding portions in order not to exceed an outer diameter specified for the jaw part tool. For this purpose, the opening in the holding portions can have a recessed shoulder against which a head of the connecting element rests within the opening. Additionally or alternatively, the head of the connecting element can be machined, e. g., finished by grinding, in order to be flush with the outer surface of the fork holder. This avoids lateral projections on the jaw part tool, which could lead to snagging or tissue injuries when used in patients.

According to yet another embodiment of the jaw part tool according to the invention, a pivot region of the jaw part portion of each arm in relation to the longitudinal axis can be delimited by a boundary wall of each recess, wherein an opening angle of each jaw part portion in relation to the longitudinal axis lies in a range extending from 0° (for closed jaw part portions) to at least 15° and at most 45°, preferably to 30°, which add up to a total opening angle between the two jaw part portions of at least 30°, at most 90°, preferably 60°. The preferred total opening angle of 60° is completely sufficient for most applications and avoids the disadvantages associated with a larger opening angle, which include increased installation space requirements and poorer force transmission.

Still further embodiments of the jaw part tool according to the invention relate to the mounting opening, formed in the control tab, for the actuating pin, which is present at the actuating portion of the arm. According to one embodiment, the mounting opening can be elongated in a direction orthogonal to the longitudinal axis and can have parallel side surfaces the spacing of which corresponds to the diameter of the actuating pin. Said elongated mounting opening is designed to allow a compensating movement of the actuating pin in the mounting opening in the direction orthogonal to the longitudinal axis. The compensating movement is necessary when the control tab is moved in its longitudinal direction in a plane with the longitudinal axis, since the actuating pin follows a circular path around the joint axis.

As an alternative to an elongated mounting opening for a compensating movement of the actuating pin, the jaw part core can be designed to allow a compensating movement of the control tab in a direction orthogonal to the longitudinal axis. For this purpose, the control tab can preferably be designed to be elastically deformable in the direction orthogonal to the longitudinal axis in order to avoid an additional joint at the connection to the force transmission means. The guide profile can be dimensioned with appropriate play for the compensating movement of the control tab. It is then possible for the mounting opening to be at least partially cylindrical with a diameter that corresponds to a diameter of the actuating pin. During force transmission in the longitudinal direction, this avoids high compression, such as occurs when the actuating pin makes line contact in the elongated mounting opening. In case of a partially cylindrical mounting bore, the cylindrical shape is interrupted on a side facing away from the joint axis, i.e., the mounting opening is open there in order to save space and to be able to place the actuating axis as far away from the joint axis as possible.

A bipolar surgical instrument as a further subject matter of the invention has an actuating means, an instrument shaft, and a jaw part tool with two, pivotally mounted arms and a force transmission means. The arms having an electrically conductive material are arranged at a distal end of the instrument shaft. The force transmission means extends movably along the longitudinal axis and through the instrument shaft and is coupled to the actuating means which is arranged at a proximal end of the instrument shaft. According to the invention, the jaw part tool of the bipolar surgical instrument is a bipolar surgical jaw part tool according to the invention in accordance with at least one of the previously described embodiments.

In one embodiment, the actuating means of the bipolar surgical instrument can be a manually operated handle, and in another embodiment, a robotically operated handle.

Another subject matter of the invention is a robotic surgical instrument system having at least one control unit, an electrosurgical generator, and a robot arm connected to the control unit, and having a bipolar surgical instrument according to the invention that is connected to the electrosurgical generator.

Further embodiments, as well as some of the advantages associated with these and other embodiments, are made apparent and better understood from the following detailed description with reference to the accompanying figures. Objects or parts thereof which are substantially the same or similar may be provided with the same reference signs. The figures are merely a schematic representation of an embodiment of the invention. The drawings, the description, and the claims contain numerous features in combination. It is understood that the features mentioned above and those to be explained below can be used not only in the combination specified in each case, but also in other combinations or on their own, without departing from the scope of the present invention.

### BRIEF DESCRIPTION OF THE DRAWINGS

In the drawings:

FIG. 1 shows a perspectival view of a jaw part tool according to an embodiment according to the invention;

FIG. 2 shows a side view of a bipolar surgical instrument according to the invention with a jaw part tool according to the invention;

FIG. 3 shows a perspectival view of the jaw part core of a jaw part tool according to the invention from a first side;

FIG. 4 shows a perspectival view of the jaw part core of FIG. 3 from the other side;

FIG. 5 shows a perspectival view of the jaw part core of FIG. 3 with an arm and the distal end of the force transmission means;

FIG. 6 shows a cross-sectional view through the jaw part tool of FIG. 1 with a cutting plane through the joint axes A, A′, which cutting plane is orthogonal to the longitudinal axis L; FIG. 7 shows a perspectival view of the jaw part core of FIG. 3 with an arm in the closed position, a first control tab, and the distal end of the force transmission means;

FIG. 8 shows a perspectival view corresponding to FIG. 7 with the arm in the open position;

FIG. 9 shows a perspectival, partially exploded view of the jaw part tool of FIG. 1;

FIG. 10 shows a perspectival detail view of the jaw part core with a first control tab according to an alternative embodiment of a jaw part tool according to the invention;

FIG. 11 shows a perspectival view of the jaw part core of FIG. 4 with two arms, a second control tab, and the force transmission means with a sleeve;

FIG. 12 shows a perspectival view corresponding to FIG. 11 with a fork holder;

FIG. 13 shows a perspectival view corresponding to FIG. 12 with a shaft connector sleeve;

FIG. 14 shows a longitudinal sectional view through the jaw part tool of FIG. 13 with a sectional plane through the longitudinal axis, which sectional plane is parallel to the joint axes A, A′; and

FIG. 15 shows a perspectival view of a robotic surgical instrument system with a bipolar surgical instrument according to the invention.

### DETAILED DESCRIPTION

The invention relates to a jaw part tool for bipolar surgical instruments with two arms that can be moved relative to one another, such as double-sided opening forceps and scissor instruments. The invention further relates to a bipolar surgical instrument equipped with a corresponding jaw part tool. The jaw part mechanism with the special, bifunctional jaw part core made of electrically insulating, i.e., non-conductive, material serves simultaneously to electrically separate the arms and to mechanically support them. This ensures that no additional insulators are required on the arms.

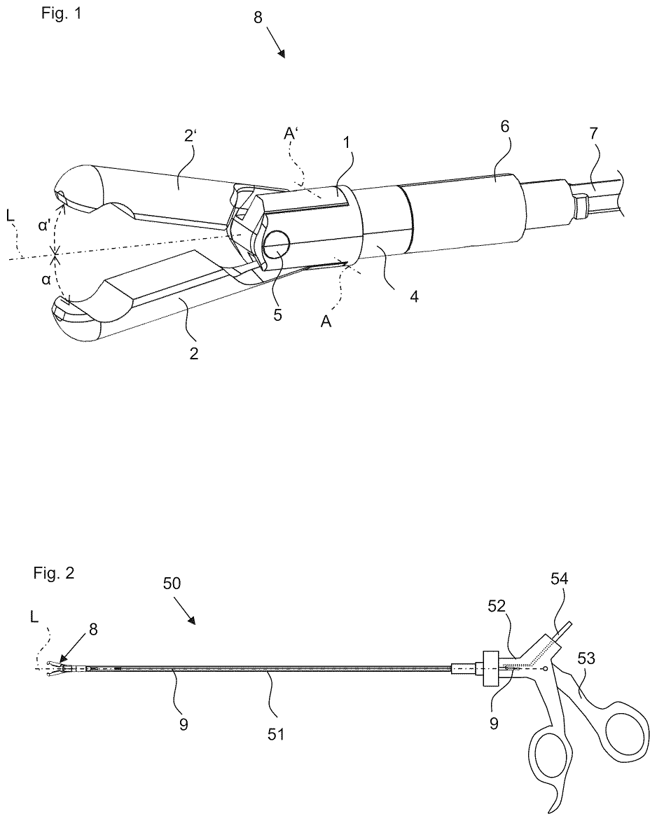

The jaw part tool 8 shown in FIGS. 1 to 14 is designed as an insert for a bipolar surgical instrument 50, as shown by way of example in FIG. 2. The jaw part tool 8, both arms 2, 2′ of which are pivotally mounted and have an electrically conductive material, is arranged at the distal end of an instrument shaft 51. A force transmission means 9 of the jaw part tool 8 is operatively connected to the arms 2, 2′ and extends through the instrument shaft 51 in a longitudinally movable manner along a common longitudinal axis L, which instrument shaft is connected at its proximal end to an actuating means 52, which in the example shown is a manually operated handle 52, which has a movable handle part 53 that is mechanically connected to the force transmission means 9 for opening and closing the arms 2, 2′, which is indicated by the dashed line. The handle 52 is furthermore equipped with a connection socket 54 for connecting the bipolar surgical instrument 50 to a generator (not shown) for supplying electrical energy. The handle 52 establishes an electrical connection between the connection socket 54 and the force transmission means 9, which is indicated by the dotted lines, in order to be able to use the arms 2, 2′ as electrodes for electrocoagulation when current is applied. As an alternative to the exemplary manually operated handle, it is also possible for a bipolar surgical instrument according to the invention to have a robotic actuating unit that is controlled by means of an input device.

The two arms 2, 2′ of the jaw part tool 8 are mounted on a jaw part core 1, which consists of an electrically insulating material. For this purpose, a plastic or ceramic material with a high CTI value is preferably chosen. For example, the jaw part core 1 can be made of a plastic with a high CTI value such as polyvinylidene fluoride PVDF (CTI value 600), e.g., milled or mass-produced by injection molding. However, it is also advantageous to mass-produce the jaw part core 1 from ceramic, thereby achieving the best mechanical, chemical, and electrical properties.

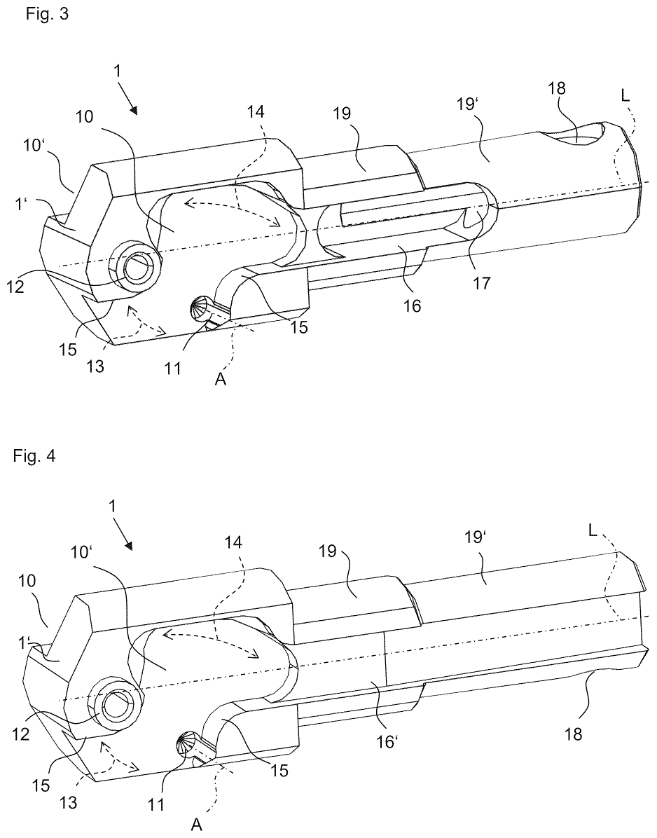

FIGS. 3 and 4 show the jaw part core 1 from both sides, which has two recesses 10, 10′, separated from one another by a core wall 1′, for mounting the arms 2, 2′. FIG. 3 shows a first recess 10 on the jaw part core 1 for rotatably mounting a first arm 2 around a first joint axis A. FIG. 4 shows the side of the jaw part core 1 with the second recess 10′, which is designed for rotatably mounting a second arm 2′ around a second joint axis A′.

The two recesses 10, 10′ are rotationally symmetrical with respect to the longitudinal axis L on opposite sides of the core wall 1′, so that the two arms 2, 2′ are identically shaped in the example shown. And because the arms 2, 2′ are completely electrically insulated from one another by the jaw part core 1, the arms 2, 2′ can be manufactured in one piece from an electrically conductive material without additional insulators.

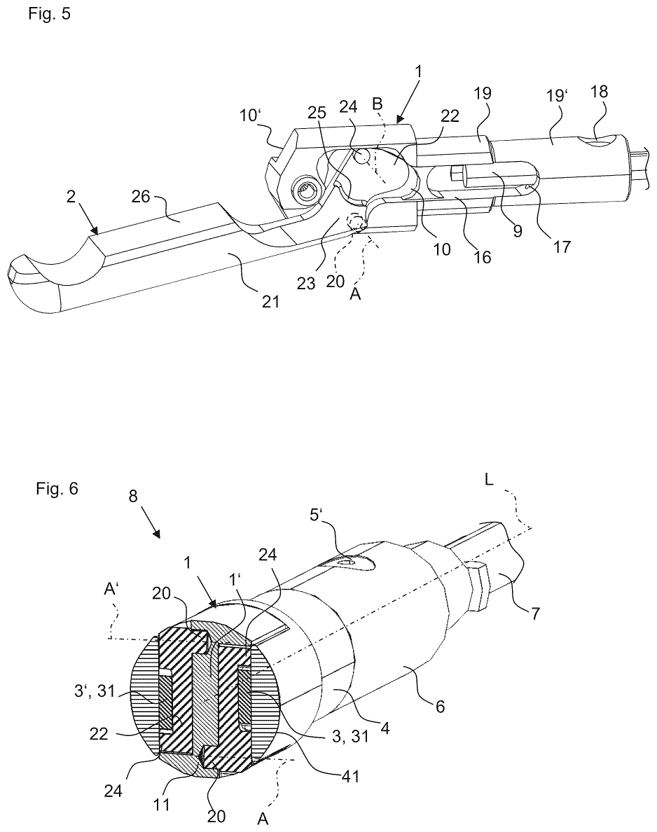

In the core wall 1′, for the two joint axes A, A′, by which each recess 10, 10′ is divided into an exit pivot region 13 and an actuation pivot region 14, a joint bushing 11 is formed. It does not penetrate the core wall 1′, so that no connection is created between the two recesses 10, 10′. The two joint axes A, A′ run orthogonally to the longitudinal axis L and are spaced apart therefrom in the radial direction relative to the longitudinal axis L, which can also be seen in FIG. 6. A joint pin 20 is rotatably received in the joint bushing 11, which joint pin each arm 2, 2′ includes. In FIG. 5, the joint pin 20 of the first arm 2, which is hidden in the illustration, is shown in dashed lines. It can be seen there that the joint pin 20 or the joint axis A formed thereby is present at an angled mounting portion 23. The latter separates a jaw part portion 21 of the arm 2, which extends through the exit pivot region 13 of the recess 10, from an actuating portion 22 of the arm 2, which is arranged in the actuation pivot region 14 of the recess 10. The jaw part portion 21 of each arm 2, 2′ has at least one functional surface 26, on the one hand for grasping, preparing, or cutting and, on the other, for electrocoagulation. The same applies to the rotatable mounting of the second arm 2′ in the second recess 10′.

The actuating portion 22 of each arm 2, 2′ serves to engage a control tab 3, 3′ connected to the force transmission means 9 for mechanically and electrically controlling the arms 2, 2′, as shown in FIGS. 7 to 11 and 14. This means that the control tab 3, 3′ ensures, on the one hand, mechanical transmission of the movement of the force transmission means 9 to the arm 2, 2′ and, on the other, the transmission of electrical energy. Therefore, the control tabs 3, 3′ are also made, preferably in one piece, from an electrically conductive material.

As shown in FIGS. 5 and 6, an actuating pin 24 is formed on the actuating portion 22 in the opposite direction to the joint pin 20, for mechanical engagement of the control tab 3, 3′ with the actuating portion 22. Said actuating pin 24 is received in a mounting opening 30 of the control tab 3, 3′. Thus, the actuating pin 24 defines an actuating axis B which is spaced from the joint axis A, A′ and runs parallel thereto. Electrical engagement of the control tab 3, 3′ with the actuating portion 22 takes place via a planar contact, for which purpose a contact portion 31 of the control tab 3, 3′ is designed to be planar in a region around the mounting opening 30. Accordingly, the actuating portion 22 is also designed to be planar in a region around the joint pin 20, so that the contact surface is as large as possible for low electrical transition or contact resistance. The respective contact surfaces of the actuating portion 22 and contact portion 31 are orthogonal to the joint axis A, A′.

Said actuating pin 24 is moved by the control tab 3, 3′ to open and close the jaw part portion 21. The further apart the joint and actuating pins 20, 24, and thus the joint and actuating axes A, A′; B, are, the better the lever ratio to the jaw part portion 21. In other words, the distance of the actuating axis B, i.e., the mechanical engagement point of the control tab 3, 3′, from the joint axis A, A′ determines the lever arm for the torque with which the arm 2, 2′ is moved around the joint axis A, A′. Therefore, the distance of the actuating pin 24 from the joint pin 20 is chosen to be as large as possible by arranging the actuating pin 24 at the edge of the actuating portion 22, at a maximum distance from the pivot axis A, A′. With a larger lever arm between the actuating pin 24 and the joint pin 20, opening and closing the jaw part portions 21 can be controlled more precisely in the application, and, with the same force applied to the force transmission means 9, a higher closing force can be achieved between the jaw part portions 21.

The planar shape of the actuating portion 22 and the distance between the joint A, A′ and the actuating axis B optimize electrical contact and mechanical engagement. Therefore, the shape and dimensions of the actuating portion 22 are matched to the shape and dimensions of the actuation pivot region 14 of the recess 10, 10′ such that the actuation pivot region 14 allows movement of the actuating portion 22 around the joint axis A, A′ within an angular range for opening and closing the jaw part portion 21 of the same arm 2, 2′. It should be taken into account that the installation space available for the recess 10, 10′ in a jaw part tool 8 for a surgical instrument 50 is often limited to a few millimeters in diameter. Therefore, deviations of the actuating portions and recesses of a jaw part tool 8 according to the invention with regard to shape and dimensions from the example shown are possible, and depend not only upon the available installation space, but also upon the function of the arms and their opening angles, wherein the aim is to achieve the largest possible lever arm between the actuating axis B and the joint axis A, A′ in the available installation space.

In the present case, the recess 10, 10′ is formed with a boundary wall 15 such that each of the two (symmetrical) arms 2, 2′ can open at an opening angle α, α′ in the range of 0° with the jaw part portions 21 closed to 30° in each case with respect to the longitudinal axis L. This results in a total maximum opening angle between the two jaw part portions 21 of 60°. This angle is sufficient or common for most jaw part tools. Although larger angles can be realized, they require more installation space and are associated with poorer force transmission, without providing any added value in most applications.

To secure both the rotatable mounting of the arms 2, 2′ in the recesses 10, 10′ and the engagement of the control tabs 3, 3′ with the arms 2, 2′, the jaw part tool 8 has a fork holder 4, as can be seen in FIGS. 1, 6, 9, 12 to 14.

The fork holder 4 is made of electrically insulating material and has two holding portions 41, 41′ on the distal side, which extend from a tube portion 44 which is offset from a proximal tube portion 43. The holding portions 41, 41′ are formed to be rotationally symmetrical around the recesses 10, 10′ for positively gripping the jaw part core 1. For this purpose, the holding portions 41, 41′ have, parallel to the core wall 1′, an inner surface, with which the holding portions 41, 41′ hold the arms 2, 2′ and control tabs 3, 3′ in the recesses 10, 10′. Since the control tabs 3, 3′ with their contact portions 31 are pressed flat against the actuating portions 22 of the arms 2, 2′, the electrical contact resistance is, advantageously, particularly low. On the distal side, the holding portions 41, 41′ are flush with the jaw part core 1.

The positive arrangement of the fork holder 4 on the jaw part core 1 is supplemented by the tube portions 43, 44, which are arranged on correspondingly shaped tube portions 19, 19′ of the jaw part core 1. This means that an inner diameter of the offset tube portion 44 of the fork holder 4 corresponds to an outer diameter of the offset tube portion 19 of the jaw part core 1, and an inner diameter of the proximal-side tube portion 43 of the fork holder 4 corresponds to an outer diameter of the proximal-side tube portion 19′ of the jaw part core 1. Also, the offset portions 44; 19 and the proximal portions 43; 19′ are each formed with corresponding lengths.

In order to form long creepage stretches in each holding portion 41, 41′, the fork holder 4 has at the free, distal end an opening 40, which, when the fork holder 4 is arranged on the jaw part core 1, receives a collar 12 corresponding in shape and dimensions, which is formed on the distal side adjacent to the recess 10, 10′ on the jaw part core 1. In order to allow the holding portions 41, 41′, when the fork holder 4 is pushed onto the jaw part core 1, to spread slightly before they snap into place with the openings 40 around the collar 12, the fork holder 4 is made of a plastic which allows elastic deformation of the holding portions 41, 41′. Thus, the fork holder 4, which is positively arranged on the jaw part core 1, forms a stable unit without any additional connecting means and secures the arms 2, 2′ against loosening. However, the fork holder 4 is not relevant for the opening mechanism of the arms 2, 2′ with the jaw part core 1 and the control tabs 3, 3′.

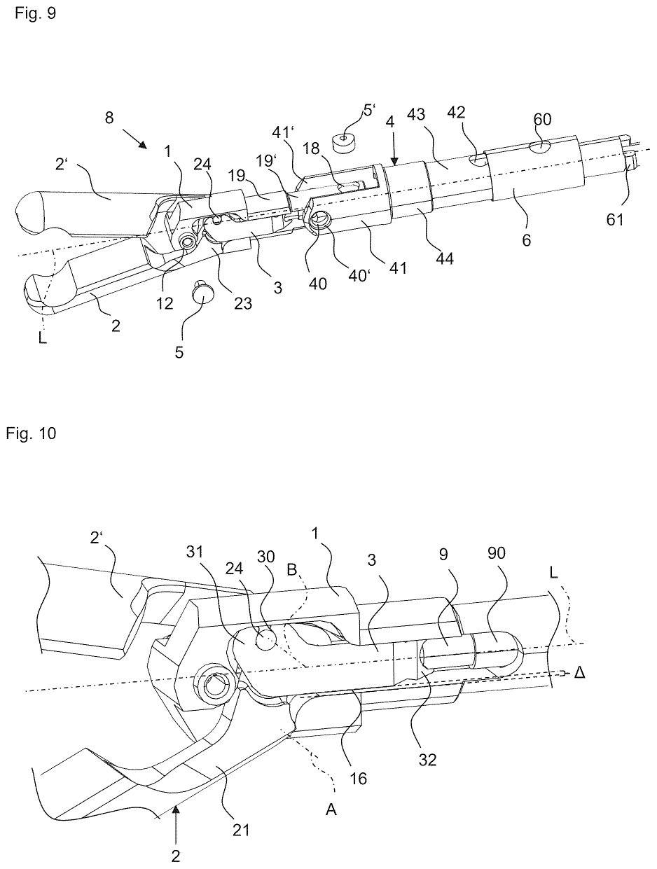

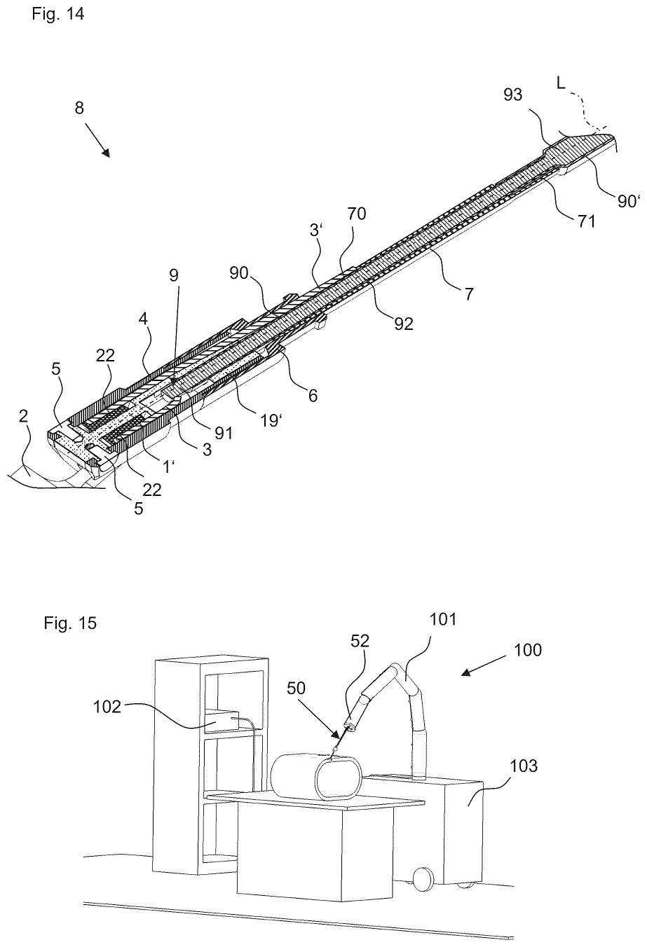

In order to further improve the lateral hold of the arms 2, 2′, the holding portions 41, 41′ are each secured with a cylindrical connecting element 5 (here, a screw). This is important in particular for scissor tools, the jaw part portions 21 of which must be guided tightly at the sides so that the tissue does not slip between the cutting edges, but is cleanly severed. The screw 5 is inserted through the opening 40 and into the collar 12 so that an offset head of the screw 5 covers the opening 40 and fixes the holding portions 41, 41′ to the jaw part core 1. In order for the screw 5 to be flush with the outer surface of the holding portions 41, 41′ on the head side, a recessed shoulder 40′ is formed in the opening 40, as can be seen in FIGS. 9, 14. Additionally or alternatively, the head of the screw 5 can be machined, e.g., finished by grinding, in order to be flush with the surface of the holding portion 41, 41′.

The fork holder 4 further has, in the proximal-side tube portion 43, a radially aligned locking opening 42, which, when positively arranged on the jaw part core 1, is aligned with a corresponding locking opening 18 in the proximal-side tube portion 19′ of the jaw part core 1. The locking openings 18, 42 are designed to receive a locking element 5′—here, locking bolt 5′—which is provided in particular for fastening a shaft connector sleeve 6. The shaft connector sleeve 6, which is designed for arrangement on the proximal-side tube portion 43 of the fork holder 4, accordingly has a locking opening 60 which, when arranged as intended, is aligned with the locking openings 42, 18 of the fork holder 4 and jaw part core 1. Here, the shaft connector sleeve 6 also has two bayonet cams 61 as shaft connecting elements, which serve for connecting to an instrument shaft 51, which for this purpose has two bayonet grooves (not shown) at its distal end. Alternative connecting elements for (releasably) connecting a jaw part tool 8 according to the invention to an instrument shaft 51 by means of a shaft connector sleeve 6 include, for example, screw, plug, and snap connections.

A jaw part tool 8 according to the invention can further have a fork holder, which differs from the example shown. For example, the fork holder could consist of two identical holder shells made of a non-elastically deformable plastic material or even ceramic. The half-shells, each of which includes one of the holding portions, positively enclose the jaw part core 1 in a corresponding manner and are held together on the distal side by the cylindrical connecting elements 5 and on the proximal side by a sleeve-for example, the shaft connector sleeve 6. The gap remaining between the holder shells would be electrically non-critical because it runs in the longitudinal direction, i.e., transversely to the relevant creepage stretches between the control tabs 3, 3′ or actuating portions 22 of the arms 2, 2′.

Another alternative provides for a continuous cylindrical connecting element (e.g., rivet) instead of the two screws 5. Since, for this purpose, a through-bore extends between the collars 12 transversely to the longitudinal axis L through the jaw part core 1, the continuous connecting element can be selected from an electrically non-conductive material in order to reduce the creepage stretch risk, and/or a distance of the collar-and thus of the through-bore—from the recess can be increased.

Additionally or as a further alternative, a material bond between the holding portions 41, 41′ and/or tube portions 43, 44 of the fork holder 4 and the jaw part core 1, e.g., by plastic welding or gluing, etc., is conceivable. The material bond means that any creepage stretch between the control tabs or the actuating portions can be completely prevented, since no liquid can penetrate into the jaw part tool. In case of purely positive fastening, a press fit or the tightening torque of the connecting element ensures that a gap between the holding portions and the jaw part core, and thus penetration of liquid, is prevented or minimized.

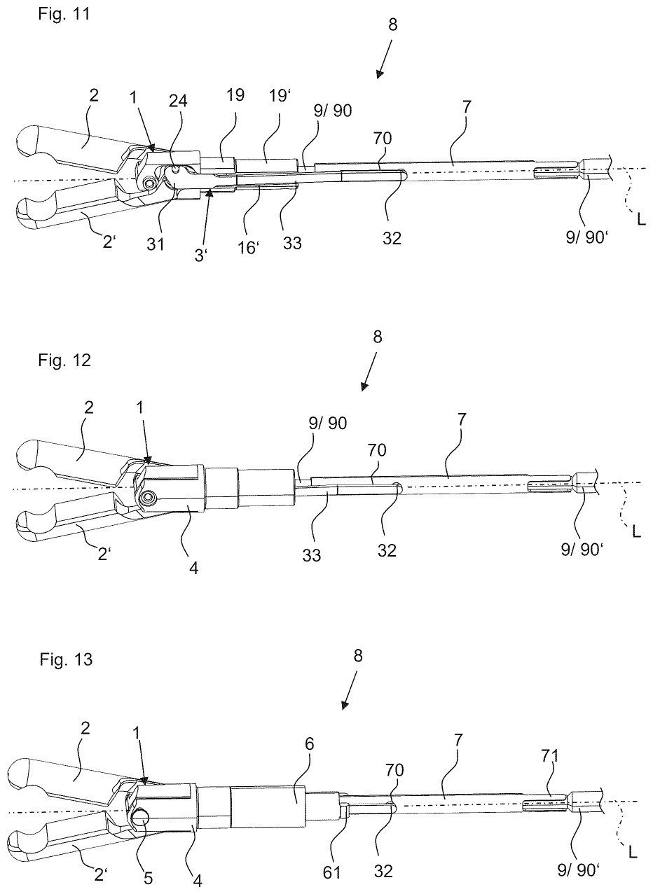

For an electrically separate connection of each control tab 3, 3′ to the force transmission means 9, which consists of an electrically conductive material, the jaw part tool 8 has a sleeve 7 as a second electrical conductor, which is partially shown in FIGS. 1, 6 and is completely shown in particular in FIGS. 11 to 14. The first control tab 3 is mechanically and electrically connected to the force transmission means 9 at a first, distal end portion 91, which is uninsulated. The second control tab 3 is mechanically and electrically connected to the sleeve 7, which is arranged on a second portion 92 of the force transmission means 9 proximal to the jaw part core 1. The second control tab 3′ is therefore longer than the first control tab 3. The second portion 92, which adjoins the distal end portion 91, is encased with an insulating layer 90, e.g., by a shrink hose. The sleeve 7 is electrically insulated from the force transmission means 9 by the insulating layer 90 and the distance to the distal end portion 91. The sleeve 7 and the force transmission means 9 thus ensure electrical conduction through the instrument shaft 51 between the control tabs 3, 3′ and the actuating means 51 with the electrical connection 54.

Mechanically, the sleeve 7, which extends on the proximal side to a third portion 93 of the force transmission means 9, which is offset from the second portion 92, is connected to the force transmission means 9, so that the movement of the force transmission means 9 in the direction of the longitudinal axis L for opening and closing the jaw part portions 21 is transmitted via both control tabs 3, 3′. In the example shown, the sleeve 7 has a distal longitudinal opening 70 which is dimensioned to receive a proximal connecting portion 32 of the second control tab 3′.

The force transmission means 9 projects with its distal end portion 91 in a longitudinally movable manner into a longitudinal bore 17 of the jaw part core 1, which extends through the proximal tube portion 19′ into the offset tube portion 19′. The distal end portion 91, to which the first control tab 3 is connected, is thus located within the longitudinal bore 17 in the region of the tube portions 19, 19′. A breakthrough is required in order for the first control tab 3 to extend from the recess 10 into the longitudinal bore 17. For this purpose, on the jaw part core 1, a first guide profile 16 for guiding the first control tab 3 is formed, which extends from the actuation pivot region 14 of the first recess 10 along the offset tube portion 19 and opens into the longitudinal bore 17 in the transition region to the proximal tube portion 19′, as can be seen in FIGS. 3, 7, 8, 10, and 14. For the second control tab 3′, the second guide profile 16′ extends from the actuation pivot region 14 of the second recess 10′ along the two tube portions 19, 19′ in order to guide the second control tab 3′ in the direction of the sleeve 7 (FIGS. 4, 11, 14). The guide directions of both guide profiles 16, 16′ are in a plane with the longitudinal axis L, so that the control tabs 3, 3′ are moved approximately parallel to the longitudinal axis L.

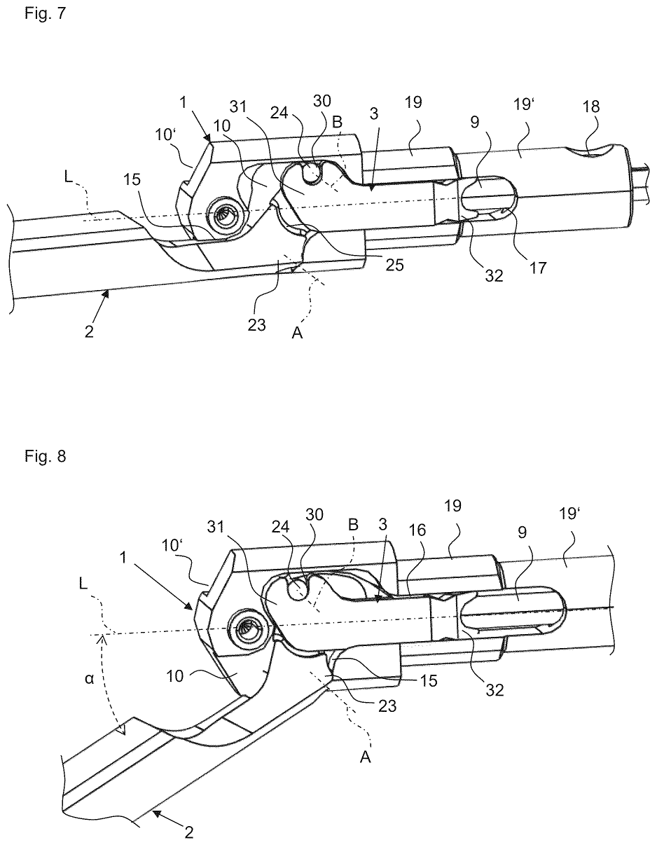

The actuating pin 24 moved by the respective control tab 3, 3′ follows a circular path around the joint axis A, A′ and therefore also performs a movement portion in a direction orthogonal to the longitudinal axis L, when opening and closing the jaw part portions 21, relative to the control tab 3, 3′ that is moved parallel to the longitudinal axis L. Therefore, the mounting opening 30 in the control tab 3, 3′ is elongated in a direction orthogonal to the longitudinal axis L, so that the actuating pin 24 can slide up and down, as can be seen in FIGS. 7 and 8. The elongated mounting opening 30, which is open outwards, i.e., on a side facing away from the longitudinal axis L, has parallel side surfaces the spacing of which corresponds to the diameter of the actuating pin 24. Force transmission in the longitudinal direction takes place between the side surface of the elongated mounting opening 30 and the cylindrical surface of the actuating pin 24.

In order to avoid the increased compression associated with such line contact, the mounting opening 30 of the control tab 3, 3′ can, in an alternative embodiment illustrated in FIG. 10, be designed cylindrically like the actuating pin 24, so as to allow for planar, circumferential force transmission. Here too, the mounting opening 30 can be open outwards, i.e., on a side facing away from the longitudinal axis L, so that the cylindrical shape of the mounting opening 30 is partially interrupted in order to be able to place the actuating pin 24 as far away as possible from the joint axis A, A′ in the available installation space. Since no compensating movement of the actuating pin 24 in the direction orthogonal to the longitudinal axis L is possible in an at least partially cylindrical mounting opening 30, the diameter of which corresponds to the diameter of the actuating pin 24, the guide profile 16, 16′ in the jaw part core 1 is formed with lateral play for the control tab 3, 3′. The control tab 3, 3′ with the actuating pin 24 can thus carry out the compensating movement in the direction orthogonal to the longitudinal axis L. The extent of such compensating movement is so small compared to the length of the control tab 3, 3′ that it can pre-form elastically and does not require a joint for connecting to the force transmission means 9. Because the guide profile 16, 16′ is designed with play, a gap Δ, which can be seen in FIG. 10, is created at the, in the figure, lower edge of the control tab 3, 3′ when the actuating pin 24 is located at the, in the figure, upper reversal point of the circular path around the joint axis A, A′. If, however, the jaw part portions 21 are maximally open or closed (not shown), the actuating pin 24 is located at the, with respect to the illustration in FIG. 10, lowest end points of its circular path, so that the control tab 3, 3′ is moved downwards, and the gap in the guide profile 16, 16′ is created at the upper edge of the control tab 3, 3′.

FIG. 15 shows a robotic surgical instrument system 100 having a bipolar surgical instrument 50. It is located at one end of a robot arm 101, which provides the actuating means 52 of the bipolar surgical instrument 50. The robot arm 101 is connected to a control unit 102 and provides the electrical connection between the electrosurgical generator 103 and the bipolar surgical instrument 50.

The drawings, the description, and the claims contain numerous features in combination. It goes without saying that the aforementioned features can be used not only in the combination specified in each case, but also in other combinations or on their own, without departing from the scope of the present invention.

The present invention provides a bipolar surgical jaw part tool 8 and a bipolar surgical instrument 50 created therewith. The jaw part tool 8 has two, pivotally mounted arms 2, 2′, which have an electrically conductive material, and a force transmission means 9, which is operatively connected to the arms 2, 2′ and is designed to provide the instrument 50 by coupling to an instrument shaft 51 and to an actuating means 52, wherein the arms 2, 2′ are arranged on the distal side of the instrument shaft 51, and the force transmission means 9 is arranged in the instrument shaft 51 so as to be movable along a longitudinal axis L and capable of being brought into engagement with the actuating means 52 on the proximal side of the instrument shaft 51. The jaw part tool 8 has a jaw part core 1 made of an electrically insulating material and two recesses 10, 10′ separated from one another by a core wall 1′, each providing a joint axis A, A′ which divides the recesses 10, 10′ into an exit and an actuation pivot region 13, 14. Each arm 2, 2′ has a mounting portion 23 between a jaw part portion 21 and an actuating portion 22, on which mounting portion the arm 2, 2′ is mounted in the recess 10, 10′ so as to be pivotable around the respective joint axis A, A′, wherein the jaw part portion 21 extends through the exit pivot region 13 and out of the recess 10, 10′, and the actuating portion 22 is arranged in the actuation pivot region 14 of the recess 10, 10′. The jaw part tool 8 has two control tabs 3, 3′, which are connected to the force transmission means 9 and each extend into the actuation pivot region 14 of the recesses 10, 10′ and there engage with the actuating portion 22 of the respective arm 2, 2′. Furthermore, a robotic surgical instrument system is disclosed.

### LIST OF REFERENCE SIGNS

1 Jaw part core
1′ Core wall
2,2 Arms
3, 3′ Control tab
4 Fork holder
5, 5′ Cylindrical connecting element, locking element
6 Shaft connector sleeve
7 Sleeve
8 Jaw part tool
9 Force transmission means
10, 10′ Recess
11 Joint bushing
12 Mounting collar
13 Exit pivot region
14 Actuation pivot region
15 Boundary wall
16, 16′ Guide profile
17 Longitudinal bore
18 Locking opening
19, 19′ Offset tube portion, proximal tube portion
20 Joint pin
21 Jaw part portion
22 Actuating portion
23 Mounting portion
24 Actuating pin
25 Boundary shoulder
26 Functional surface
30 Mounting opening for actuating pin
31 Contact portion
32 Connecting portion
33 Extension portion
40, 40′ Receiving opening, recessed shoulder
41, 41′ Holding portion
42 Locking opening
43, 44 Proximal tube portion, offset tube portion
50 Bipolar surgical instrument
51 Instrument shaft
52 Actuating means/handle
53 Movable handle part
54 Connection socket
60 Locking opening
61 Shaft connecting element/bayonet cam
70 Distal longitudinal opening
71 Proximal longitudinal tab
90, 90′ Insulating layers
91, 92, 93 Distal end portion/first portion, second portion, third/offset portion
100 Robotic surgical instrument system
101 Robot arm
102 Control unit
103 Electrosurgical generator
A, A′ Joint axis
B Actuating axis
L Longitudinal axis
α, α′ Opening angle
Δ Gap

1. A bipolar surgical jaw part tool comprising:
two, pivotally mounted arms, which have an electrically conductive material, and a force transmission device, which is operatively connected to the arms, wherein the jaw part tool is configured to provide a bipolar surgical instrument by coupling to an instrument shaft and to an actuating device, wherein the arms are arrangeable at a distal end of the instrument shaft, and the force transmission device is arrangeable in the instrument shaft so as to be movable along a common longitudinal axis and configured to be brought into engagement with the actuating device at a proximal end of the instrument shaft,
wherein
the jaw part tool has a jaw part core made of an electrically insulating material and two recesses separated from one another by a core wall, each configured to provide a joint axis which divides the recesses into an exit pivot region and an actuation pivot region, wherein each arm has a mounting portion between a jaw part portion and an actuating portion, on which mounting portion the arm is mounted in the recess so as to be pivotable around the respective joint axis, wherein the jaw part portion extends through the exit pivot region and out of the recess, and the actuating portion is arranged in the actuation pivot region of the recess, and wherein the jaw part tool has two control tabs which are connected to the force transmission device and each extend into the actuation pivot region of the recesses and there engage with the actuating portion of the respective arm.

2. The jaw part tool according to claim 1, wherein
the jaw part tool has a fork holder made of an electrically insulating material, which has at least one tube portion on the proximal side for arrangement on at least one tube portion of the jaw part core, and two holding portions on the distal side which are configured to positively grip the jaw part core around the recesses.

3. The jaw part tool according to claim 1, wherein
the joint axes of the arms in the recesses of the jaw part core are each formed by a joint pin rotatably mounted in a joint bushing, wherein the joint bushings are formed in the recesses and the joint pins are formed on the arms, or the joint pins are formed on the recesses and the joint bushings are formed in the arms,
and wherein the recesses are formed to be rotationally symmetrical with respect to the longitudinal axis on the jaw part core on opposite sides of the core wall, and wherein the two joint axes run orthogonally to the longitudinal axis and are radially spaced therefrom.

4. The jaw part tool according to claim 1, wherein
the engagement of the control tab with the actuating portion is provided by an actuating pin received in a mounting opening, which actuating pin defines an actuating axis that is spaced from and parallel to the joint axis, wherein the actuating pin is formed on the actuating portion and the mounting opening is formed in the control tab, or the mounting opening is formed in the actuating portion and the actuating pin is formed on the control tab.

5. The jaw part tool according to claim 1, wherein
each arm is made in one piece from the electrically conductive material, wherein the jaw part portion has at least one functional surface, and the actuating portion is made of an electrically conductive material for planar contact with a planar contact portion of the control tab, wherein respective contact surfaces of the actuating portion and contact portion are orthogonal to the joint axis.

6. The jaw part tool according to claim 1, wherein
the force transmission device has a sleeve for connecting to the control tabs and is divided into a first portion, which is a distal, uninsulated end portion, and into a second, circumferentially insulated portion, which adjoins the distal end portion and is offset on the proximal side from a third, circumferentially insulated portion of the force transmission means, wherein
the sleeve is arranged on the second portion and extends to the third portion, and
a first control tab is electrically and mechanically connected to the distal, uninsulated end portion, and
a second control tab, which is longer than the first control tab, is electrically and mechanically connected to the sleeve.

7. The jaw part tool according to claim 2, wherein
the at least one tube portion of the jaw part core has a longitudinal bore into which the force transmission device extends in a longitudinally movable manner.

8. The jaw part tool according to claim 7, wherein
the jaw part core has a guide profile for each control tab, in which the respective control tab is guided and movable in a direction which lies in a plane with the longitudinal axis, wherein
a first guide profile which is configured to guide the first control tab, which is connected to the distal end portion, extends from the actuation pivot region of a first recess along the at least one tube portion and ends in front of a proximal end of the at least one tube portion and opens into the longitudinal bore, and
a second guide profile which is configured to guide the second control tab, which is connected to the sleeve, extends from the actuation pivot region of a second recess to the proximal end of the at least one tube portion.

9. The jaw part tool according to claim 1, wherein
the jaw part tool has a shaft connector sleeve which is connected to the jaw part core on the proximal side and has at least one shaft connecting element which is configured for connecting to the instrument shaft.

10. The jaw part tool according to claim 9, wherein
the shaft connector sleeve configured for arrangement on a proximal tube portion of the fork holder, which is arranged on a proximal tube portion of the jaw part core, wherein the shaft connector sleeve and the proximal tube portion of the fork holder and the proximal tube portion of the jaw part core each have a radially aligned, mutually aligned locking opening in which a locking element is arranged that connects the shaft connector sleeve and the fork holder and the jaw part core.

11. The jaw part tool according to claim 2, wherein
the two holding portions of the fork holder each have an opening, which corresponds in terms of shape and dimensions to a collar, which is formed on the jaw part core adjacent to the respective recess, wherein
the holding portions are elastically deformable, or
the fork holder is divided lengthwise into two holder shells, each of which has one of the holding portions and is held together on the proximal side by a sleeve.

12. The jaw part tool according to claim 2, wherein
the fork holder is connected to the jaw part core at the two holding portions by at least one cylindrical connecting element, or the two holding portions and/or the at least one tube portion are/is materially bonded to the jaw part core by a welded or adhesive connection.

13. The jaw part tool according to claim 1, wherein
a pivot region of the jaw part portion of each arm 29 in relation to the longitudinal axis is delimited by a boundary wall of each recess, wherein an opening angle (α, α′) of each jaw part portion in relation to the longitudinal axis lies in a range extending from 0° to at least 15° and at most 45°.

14. The jaw part tool according to claim 4, wherein
the mounting opening for the actuating pin is
an elongated mounting opening, in a direction orthogonal to the longitudinal axis, with parallel side surfaces, the spacing of which corresponds to a diameter of the actuating pin, wherein the elongated mounting opening configured to allow a compensating movement of the actuating pin in the mounting opening in the direction orthogonal to the longitudinal axis, or
an at least partially cylindrical mounting opening, the diameter of which corresponds to a diameter of the actuating pin, wherein the jaw part core is configured to allow a compensating movement of the control tab in a direction orthogonal to the longitudinal axis, wherein the control tab is configured to be elastically deformable in the direction orthogonal to the longitudinal axis.

15. A bipolar surgical instrument comprising:
an actuating device,
an instrument shaft, and
a jaw part tool with two, pivotally mounted arms and a force transmission device, wherein the arms, which have an electrically conductive material, are arranged at a distal end of the instrument shaft, and the force transmission device extends movably along the longitudinal axis and through the instrument shaft and is coupled to the actuating means which is arranged at a proximal end of the instrument shaft, and
the jaw part tool is a bipolar surgical jaw part tool according to claim 1.

16. A robotic surgical instrument system comprising:
at least one control unit,
an electrosurgical generator, and
a robot arm connected to the control unit, and having a bipolar surgical instrument that is connected to the electrosurgical generator, and
the bipolar surgical instrument is a bipolar surgical instrument according to claim 15.
